(19) United States
(12) Patent Application Publication
(10) Pub. No.: US 2024/0398357 A1
(43) Pub. Date: Dec. 5, 2024

(54) MEDICAL DEVICE ADJUSTING ACOUSTIC SIGNAL OUTPUT VOLUME AND METHOD OF ADJUSTING THE ACOUSTIC SIGNAL OUTPUT VOLUME OF A MEDICAL DEVICE

(71) Applicant: OLYMPUS WINTER & IBE GMBH, Hamburg (DE)

(72) Inventor: Stefan DIETRICH, Potsdam (DE)

(73) Assignee: OLYMPUS WINTER & IBE GMBH, Hamburg (DE)

(21) Appl. No.: 18/676,980

(22) Filed: May 29, 2024

Related U.S. Application Data
(60) Provisional application No. 63/470,327, filed on Jun. 1, 2023.

(51) Int. Cl. A61B 5/00; G06F 3/16

(52) U.S. Cl. CPC A61B 5/7415; G06F 3/165

(57) ABSTRACT

A medical device, including: an acoustic signal output device; a control unit configured to cause the acoustic signal output device to output acoustic signals; and a sensor. The medical device is further characterized in that the control unit is configured to receive signals from the sensor and to adjust the signal output volume at which the acoustic signal output device outputs acoustic signals based at least in part on the sensor signal. The invention further relates to a method for adjusting the acoustic signal output volume of a medical device.

The invention disclosed in the following relates to a medical device, comprising: an acoustic signal output device; a control unit configured to cause the acoustic signal output device to output acoustic signals; and a sensor. The invention further relates to a method for adjusting the acoustic signal output volume of a medical device.

In the present disclosure, the term “medical device” is used to describe various devices used for performing medical functions on a patient. In particular, the term is used for describing advanced medical devices like electrosurgical energy generators, surgical laser sources, lithotripters, endoscope camera controllers, or auxiliary medical devices like irrigation pumps, suction pumps, insufflators, smoke evacuators, or the like.

Electrosurgery generally refers to a group of medical procedures in which a radio frequency (RF) alternating electrical current is employed to achieve a therapeutic effect such as a coagulation, ablation, fulguration, desiccation or cutting of tissue. Electrosurgical currents are applied to the target tissue through electrode pads arranged on the patient's body and through electrosurgical instruments comprising contact surfaces, e.g. electrosurgical forceps with jaws or cutting blades as electrical contact surfaces. The electrosurgical instruments and pads are electrically connected to an electrosurgical generator which provides RF energy when caused to do so. The term “electrosurgery” also encompasses indirect applications of electrosurgical currents, like ultrasonic surgery, where electrosurgical currents are converted into ultrasonic vibrations, or microwave surgery, where electrosurgical currents are converted into electromagnetic waves.

Many medical devices of the aforementioned types are designed for use in an operation theatre, and typically comprise an output device for outputting acoustic signals to the operating personnel used to issue warnings or to draw attention to certain aspects of the procedure, e.g. the achievement of a specific therapeutic effect or a malfunction or failure of a piece of equipment during the medical procedure. The output volume of the acoustic signal can be adjusted on the medical device, e.g. through buttons or knobs on a front or backside of the device or through a graphical user interface (GUI) menu. An increase in volume can be necessary e.g. in case of increasing ambient acoustic noise in the operating theatre in order to improve the perceptibility of the acoustic signal for the operating physician.

However, the adjustment of the volume during an ongoing procedure comes with certain difficulties. As there is typically just one staff member specifically tasked to adjust settings at certain medical devices, that person necessarily has to suspend the current task in order to adjust the volume of the acoustic output signal. In case the medical device is placed in a ceiling-mounted supply unit an adjustment knob disposed on a rear side of the generator may, furthermore, be difficult to reach. If otherwise an adjustment of the volume is achieved via the medical device's GUI, a different menu may have to be selected first leaving the menu currently used for adjustment of different settings. Additionally, an adjustment of the volume through a GUI requires more time.

Therefore, it is an object of the invention to provide a medical device and a method for adjusting a medical device which are improved with respect to the aforementioned difficulties.

According to a first aspect of the invention, the object is achieved by a medical device, comprising: an acoustic signal output device; a control unit configured to cause the acoustic signal output device to output acoustic signals; and a sensor, wherein the generator is further characterized in that the control unit is configured to receive signals from the sensor and to adjust the signal output volume at which the acoustic signal output device outputs acoustic signals based at least in part on the sensor signal.

A medical device according to the invention is capable of adjusting the output volume automatically. An interaction with a staff member is not necessarily needed. Consequently, neither a manipulation of buttons, knobs or the like on the front or back side of the generator nor of a GUI is required. Therefore, the medical personnel is not distracted from their actual task. An automatic adjustment of the volume may also be carried out quicker than a manual adjustment allowing a more immediate reaction to sudden changes in volume requirements.

Acoustic signals to be output may include beeps, chords, continuous signals with varying pitch to indicate therapy progress, spoken messages, or the like. Such messages may indicate e.g. status messages related to an ongoing electrosurgical procedure as for example an achieved therapeutic effect, malfunctions within an electrosurgical system, an incompatibility of system components or settings, or the like. The invention is, however, not limited to these messages but relates to all information acoustically output by the medical device.

The sensor signal provides information which allows the control unit to determine that an adjustment of the volume is appropriate. The sensor may include a microphone. The sensor may include a plurality of sensors, e.g. one on a front side of the medical device, and one on a rear side of the medical device.

The acoustic signal output device may be a speaker or a comparable electroacoustic transducer converting electrical signals to sound. Alternatively, the acoustic signal output device may also be an interface, e.g. a socket, to which an external speaker or the like may be connected. An interface with an external speaker may be wired or wireless.

Additionally to the sensor signal, the output volume may depend on or may be adjusted following other factors, e.g. the type or degree of criticality of the conveyed information or on information input through knobs, switches, a GUI and the like by a user.

The sensor may be a sensor for sensing ambient acoustic noise. The control unit may be configured to increase the signal output volume with rising ambient noise level. The control unit may be configured to reduce the signal output with sinking ambient noise level. An increase or change in ambient noise is a driving factor for the need to adjust the output volume of acoustic signals. Therefore, a sensor suitable for detecting this value provides a sensor signal which may be comparably easily interpreted by the control unit. An automatic adjustment of the output volume with increasing ambient noise releases a user from manually adjusting the output volume in the majority of situation in which this may be necessary.

The control unit may be configured to analyse a spectrum of ambient noise using the sensor signal and to adjust the signal output volume of the acoustic signal output device based at least in part on the analysis of the spectrum. The spectrum may represent a relation between the energy and the frequency of the recorded signal. Instead of simply adjusting the volume based on the overall energy of the recorded noise, the energy in a particular spectral region or the distribution of the energy between different regions may provide more insight into the necessity to adjust the output volume.

Preferably, the control unit may be configured to adjust the signal output volume of the acoustic signal output device based on a spectral distance between a signal to be output and a local spectral maximum of the ambient noise. For example, a larger spectral distance may indicate that only a minor adjustment or increase in output volume is necessary whereas a lower spectral distance may indicate that a more significant adjustment is appropriate.

In some embodiments, the control unit may be configured to adjust the signal output volume of the acoustic signal output device based at least in part on the sensor signal received over a predetermined or predeterminable period of time. An adjustment of the output volume may be suitable following a permanent increase or decrease of the ambient noise level or at least following a change in noise level being permanent in the near term. In contrast, immediately and repeatedly adjusting the output volume to a temporary or short term change in ambient noise, e.g. due to a single noise-producing event, may be perceived as disturbing. The average noise level over a period of time may be one possibility to derive a suitable output volume. Alternatively or additionally, short noise-peaks within the period may be disregarded or weighted less. Maximum and minimum noise levels within a period of time may also indicate a necessary adjustment of the output volume.

In a further embodiments, the electrosurgical generator may comprise an input device, and the control unit may be configured to adjust the signal output volume at which the acoustic signal output device outputs acoustic signals also based at least in part on data input via the input device. Especially, the input device may comprise one or more of a button, a switch, a knob, a rotary switch, a slider or a graphical user interface. The graphical user interface may include a touch panel or a touch screen. In such an embodiment, conventional means for adjusting the signal output volume may be used to set a base signal output volume. An automatic adjustment based on the sensor signal may then be applied around the base volume.

According to a second aspect of the invention, the object is achieved by a method for adjusting the acoustic signal output volume of a medical device, the medical device comprising an acoustic signal output device; a control unit configured to cause the acoustic signal output device to output acoustic signals; and a sensor, the method comprising the steps of: Receiving signals from the sensor, and adjusting by the control unit the signal output volume at which the acoustic signal output device outputs acoustic signals based at least in part on the sensor signal. Regarding the advantages and the technical effects of the method according to the invention reference is made to the aforementioned considerations.

Further, the step of receiving signals may comprise receiving signals representing the sensed ambient noise. In some embodiments, the step of adjusting the signal output volume may comprise increasing the signal output volume with rising ambient noise level. The method may additionally comprise the step of analysing a spectrum of the ambient noise using the sensor signal; and the step of adjusting the acoustic signal output volume may comprise adjusting the signal output volume of the acoustic signal output device based at least in part on the analysis of the spectrum. In some embodiments, the step of adjusting the signal output volume may comprise adjusting the signal output volume of the acoustic signal output device based on a spectral distance between a signal to be output and a local spectral maximum of the ambient noise. The step of adjusting the signal output volume may comprise adjusting the signal output volume of the acoustic signal output device based at least in part on the sensor signal received over a predetermined or predeterminable period of time. All these embodiments may have technical effects and advantages as discussed above.

In the following, an embodiment of the invention is described with reference to illustrative schematic drawings. In this connection, the embodiments illustrated below are merely intended to contribute to a better understanding of the invention without limiting it. While the described embodiments relate to electrosurgical systems, the invention may likewise be applied with any other medical device or medical device system. The figures show:

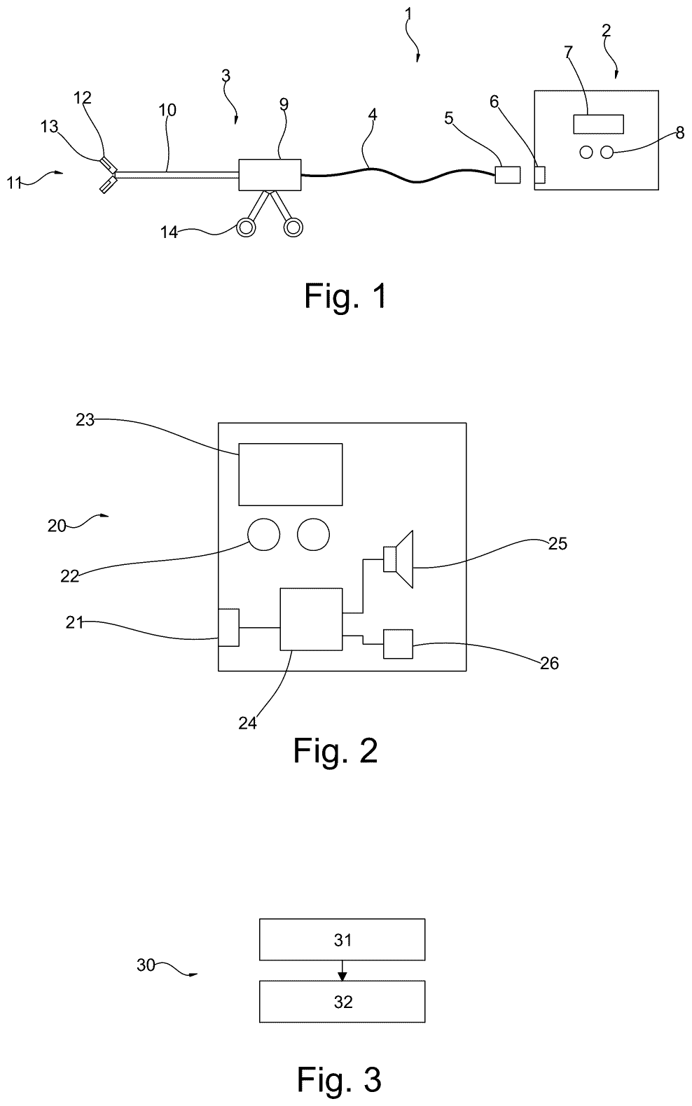

FIG. 1: An electrosurgical system;

FIG. 2: An electrosurgical generator according to the invention;

FIG. 3: A method according to the invention.

FIG. 1 shows an electrosurgical system 1 in which an electrosurgical generator according to the invention can be operated. The electrosurgical system comprises an electrosurgical generator 2 and an electrosurgical instrument 3. In operation, generator 2 and instrument 3 are connected, e.g. through a cable 4 and a plug 5 and socket 6 connector as depicted in FIG. 1.

The electrosurgical generator 2 provides RF therapy currents to the connected instrument, e.g. to instrument 3. The delivery of RF current may be initiated e.g. through operating elements disposed on the instrument 3 or non-depicted pedals operated by a physician. The electrosurgical generator 2 may further serve to receive data from the instrument 3 or commands input through the instrument's operating elements.

The electrosurgical generator 2 further comprises a display 7 and operating elements 8. The display 7 serves to output information to a user. Such information may comprise e.g. a current operating status or warning or failure messages. Through the operating elements 8 a user can for example adjust certain operating parameters or make settings to the system. It may be possible to enter a menu of settings of the generator 2 through the operating elements 8 which is then output on the display 7. Settings to be adjusted may also include the output volume of an acoustic output signal. The display may also be a touch display which can directly receive and process user inputs.

The instrument 3 is shown as a bipolar electrosurgical coagulation forceps. It includes a main body 9 and an elongated shaft 10. At a distal end of the shaft 10 a forceps 11 with movable jaws 12 is disposed. The jaws 12 each carry a treatment electrode 13. Two handle levers 14 are further arranged on the main body 9 which are movable relative to each other for actuating the jaws 12.

Electrosurgical instruments and electrodes of other or the same design can also be connected alternatively or simultaneously to an electrosurgical generator according to the invention.

FIG. 2 is an illustration of an electrosurgical generator 20 according to the invention. Like the generator 2 of FIG. 1, the electrosurgical generator 20 comprises a socket 21 for connecting an electrosurgical instrument, e.g. the instrument 3. Although just one socket 21 is illustrated, there may be more than a single socket configured to receive connection plugs for connecting alternative or additional instruments or electrosurgical return electrode pads. The generator 20 also comprises optional operating elements 22 and a display 23 which serve to adjust operating settings and display information as described above.

Furthermore, the generator 20 includes a control unit 24 and an acoustic signal output device 25. The control unit is configured to cause the acoustic signal output device to output acoustic signals. Acoustic signals to be output may include tones or spoken messages or the like and relate to the medical procedure executed or system warnings. However, the invention is not limited to these acoustic messages but rather includes all acoustic outputs typically generated by an electrosurgical generator. Accordingly, the acoustic signal output device 25 may be or include any kind of electroacoustic transducer, e.g. a speaker, converting an output signal to a sound. Alternatively or additionally, the acoustic signal output device may also be or include an interface, e.g. a socket, to which an external speaker or the like may be connected.

The electrosurgical generator 20 comprises a sensor 26. The sensor 26 is connected to the control unit 24 for providing a sensor signal. The control unit 24 is configured to receive the signals from the sensor 26 and to adjust the signal output volume at which the acoustic signal output device 25 outputs acoustic signals based at least in part on the sensor signal from sensor 26. The electrosurgical generator 20 according to the invention is, thus, capable of adjusting the output volume automatically. An interaction with a user is not necessarily needed.

The sensor signal provides information which allows the control unit 24 to determine whether an adjustment of the acoustic signal output volume is appropriate. The sensor 26 may be a sensor for sensing ambient noise. The sensor 26 may be or include a microphone. The control unit 24 may then adjust the output volume based at least in part on the ambient noise in the operation theatre. More precisely, the control unit 24 may be configured to increase the signal output volume with rising ambient noise level. In this case, it can be ensured that acoustic signals can still be heard by staff members even in a loud environment.

The control unit 24 may also be configured to analyse a spectrum of the noise recorded by sensor 26, wherein the spectrum represents a relation between the energy and the frequency of the recorded signal. An adjustment of the output volume may then be based on the energy of the recorded noise in a particular spectral region or the distribution of the energy between different regions. For example, the desired output volume may be determined based on a spectral distance between an acoustic signal to be output and a local spectral maximum of the ambient noise.

When determining the output signal volume, the control unit 24 may also take into account the signal received from the sensor 26 over a period of time. An adjustment of the output volume may then follow a permanent increase in ambient noise in contrast to adapting to temporary or single-event noises. Short noise-peaks may also be disregarded completely or weighted less.

Additionally to the sensor signal, the output volume may also depend on or may be adjusted following other factors, e.g. the type or degree of criticality of the conveyed information or on information manually input by a user. For example, via operating elements 22 and display 23 it may be possible to manually adjust a base output volume of the acoustic output signal. The generator 20 may also comprise knobs, switches, a GUI and the like not illustrated in FIG. 2, enabling a user to adjust the base output volume. An automatic adjustment of the output volume based on the sensor signal may then be applied around the base volume.

FIG. 3 illustrates a method 30 for adjusting the acoustic signal output volume of an electrosurgical generator comprising an acoustic signal output device, a control unit, and a sensor. The generator may for example be the generator 2 of FIG. 1 or the generator 20 of FIG. 2. The method starts at step 31 with receiving signals from the sensor. The sensor may be sensing ambient noise such that information on the ambient noise is contained in the received signal.

In a second step at 32, the control unit adjusts the signal output volume at which the acoustic signal output device outputs acoustic signals based at least in part on the sensor signal. The acoustic signal output volume may therein be increased with a rising ambient noise level.

The method may further comprise a step of analysing a spectrum of the ambient noise and the step 32 of adjusting the acoustic signal output volume may comprise adjusting the signal output volume of the acoustic signal output device based at least in part on the analysis of the spectrum. Especially, the signal output volume of the acoustic signal output device may be based on a spectral distance between an acoustic signal to be output and a local spectral maximum of the ambient noise. In doing so, the step 32 of adjusting the acoustic signal output volume may also take into account the sensor signal received over a predetermined or predeterminable period of time in order to follow a permanent increase or decrease of the ambient noise level instead of repeatedly reacting to temporary or short term changes in ambient noise level.

1-14. (canceled)

15. A medical device, comprising:
an acoustic signal output device;
a control unit configured to cause the acoustic signal output device to output acoustic signals; and
a sensor,
wherein
the control unit is configured to receive signals from the sensor and to adjust the signal output volume at which the acoustic signal output device outputs acoustic signals based at least in part on the sensor signal.

16. The medical device of claim 15, wherein the sensor is a sensor for sensing ambient acoustic noise.

17. The medical device of claim 16, wherein the control unit is configured to increase the signal output volume with rising ambient noise level, and/or to reduce the signal output volume with sinking ambient noise level.

18. The medical device of claim 15, wherein the control unit is configured to analyse a spectrum of the sensor signal and to adjust the signal output volume of the acoustic signal output device based at least in part on the analysis of the spectrum.

19. The medical device of claim 18, wherein the control unit is configured to adjust the signal output volume of the acoustic signal output device based on a spectral distance between the signal to be output and a local spectral maximum of the ambient noise.

20. The medical device of claim 15, wherein the control unit is configured to adjust the signal output volume of the acoustic signal output device based at least in part on the sensor signal received over a predetermined or predeterminable period of time.

21. The medical device of claim 15, wherein the electrosurgical generator comprises an input device, and the control unit being configured to adjust the signal output volume at which the acoustic signal output device outputs acoustic signals also based at least in part on data input via the input device.

22. The medical device of claim 15, wherein the input device comprises one or more of a button, a switch, a knob, a rotary switch, a slider or a graphical user interface.

23. A method for adjusting the acoustic signal output volume of a medical device, the medical device comprising an acoustic signal output device; a control unit configured to cause the acoustic signal output device to output acoustic signals; and a sensor, the method comprising the steps of:
receiving signals from the sensor, and
adjusting by the control unit the signal output volume at which the acoustic signal output device outputs acoustic signals based at least in part on the sensor signal.

24. The method of claim 23, wherein the step of receiving signals comprises receiving signals representing the sensed ambient acoustic noise.

25. The method of claim 24, wherein the step of adjusting the acoustic signal output volume comprises increasing the signal output volume with rising ambient noise level, and/or reducing the signal output volume with sinking ambient noise level.

26. The method of claim 23, additionally comprising the step of analysing a spectrum of the sensor signal; and wherein the step of adjusting the acoustic signal output volume comprises adjusting the signal output volume of the acoustic signal output device based at least in part on the analysis of the spectrum.

27. The method of claim 26, wherein the step of adjusting the acoustic signal output volume comprises adjusting the signal output volume of the acoustic signal output device based on a spectral distance between the signal to be output and a local spectral maximum of the ambient noise.

28. The method of claim 23, wherein the step of adjusting the acoustic signal output volume comprises adjusting the signal output volume of the acoustic signal output device based at least in part on the sensor signal received over a predetermined or predeterminable period of time.
